(19) United States
(12) Patent Application Publication
(10) Pub. No.: US 2013/0226006 A1
(43) Pub. Date: Aug. 29, 2013

(54) ULTRASONIC PROBE

(71) Applicant: Kentaro Tsuzuki, Nasushiobara-shi (JP)

(72) Inventor: Kentaro Tsuzuki, Nasushiobara-shi (JP)

(73) Assignees: TOSHIBA MEDICAL SYSTEMS CORPORATION, Otawara-shi, Tochigi (JP); KABUSHIKI KAISHA TOSHIBA, Tokyo (JP)

(21) Appl. No.: 13/883,922

(22) PCT Filed: Jun. 7, 2012
(86) PCT No.: PCT/JP2012/064629

§ 371 Date: May 7, 2013

(30) Foreign Application Priority Data
Jun. 8, 2011 (JP) 2011-128057

(51) Int. Cl. A61B 8/00

(52) U.S. Cl. CPC A61B 8/4494
USPC 600/459

(57) ABSTRACT

An ultrasonic probe including an ultrasonic transducer, an electrode extraction layer, and a low acoustic impedance matching layer. The ultrasonic transducer includes a plurality of elements arranged with predetermined spacing. The electrode extraction layer is electrically connected to the ultrasonic transducer. The low acoustic impedance matching layer is provided on the electrode extraction layer, having lower acoustic impedance than the ultrasonic transducer, wherein a plurality of grooves are shaped on the surface of the electrode extraction layer side in parallel to the element array direction. The ultrasonic probe prevents resolving power deterioration in ultrasonic images that may further extract electrodes of an ultrasonic transducer with high reliability.

### FIELD OF THE INVENTION

The embodiment of the present invention relates to an ultrasonic probe.

### BACKGROUND OF THE INVENTION

Ultrasonic diagnostic equipment exists that scans the inside of a subject using ultrasonic waves and images the internal state of said subject based on received signals generated by reflected waves from inside the subject.

Ultrasonic diagnostic equipment such as this transmits ultrasonic waves from an ultrasonic probe to inside the subject, receives reflected waves generated by the non-conformance of acoustic impedance inside the subject, and generates received signals. The ultrasonic probe comprises several micro-oscillators that generate ultrasonic waves by oscillating based on transmitted signals and generate received signals by receiving reflected waves in an array in the scanning direction. Furthermore, the micro-oscillator may be referred to as an element. Moreover, micro-oscillators arranged in arrays may be referred to as an ultrasonic transducer.

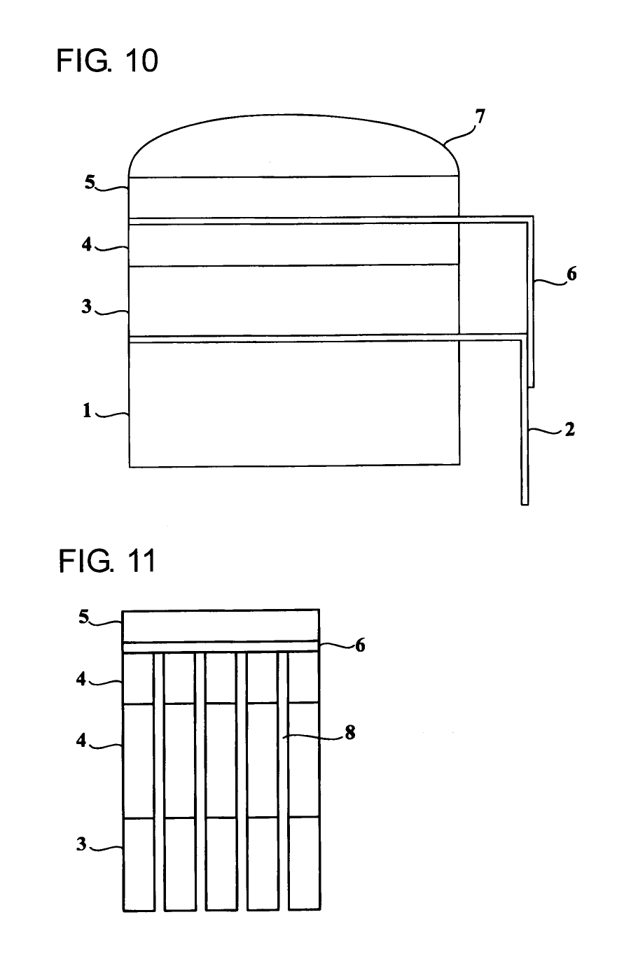

A fundamental configuration of the ultrasonic probe is described with reference to FIG. 10. FIG. 10 is a fundamental configuration of the ultrasonic 1D-array probe. As shown in FIG. 10, the ultrasonic probe comprises: an ultrasonic transducer 3 generating ultrasonic waves, a high acoustic impedance matching layer (high AI matching layer) 4 that eases the unconformity of the acoustic independence between the ultrasonic transducer and a living body from the ultrasonic transducer 3 towards the living body contact surface side, an upper surface electrode extracting layer 6, a low acoustic impedance matching layer (low AI matching layer) 5, and an acoustic lens 7 that converges ultrasonic waves. Moreover, there exists an undersurface electrode extraction layer 2 and a rear material 1 from the ultrasonic transducer 3 to the cable side (opposite side of the living body contact side). Here, an upper surface electrode is determined as a GND (ground).

The high AI matching layer 4 and the low AI matching layer 5 are established with 2 to 3 layers from the ultrasonic transducer 3 in the living organism by gradually decreasing the acoustic impedance. ¼ of a wavelength λ is widely used as the thickness of each acoustic matching layer 4 and 5. Here, the wavelength λ is the wavelength of ultrasonic waves transmitting each acoustic matching layer 4 and 5. Generally, the high AI matching layer 4 is hard with machinability, so in order to reduce acoustic coupling with the adjacent element, when the ultrasonic transducer 3 is divided, the high AI matching layer 4 is also divided at the same time. Meanwhile, the low AI matching layer 5 cannot sufficiently reduce the shape ratio (w/t) due to slow sound velocity. Thereby, the following two methods are performed. Furthermore, w and t each indicate the width and thickness of the low AI matching layer 5.

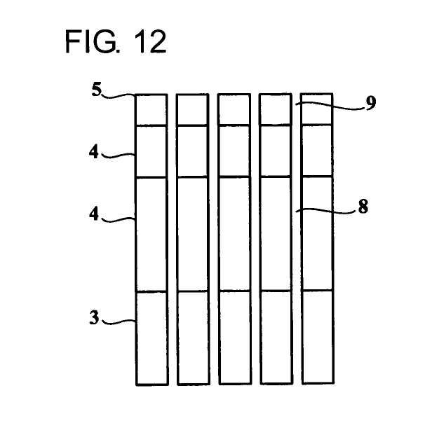

The first method involves layering the low AI matching layer 5 with rubber materials like a sheet. FIG. 11 is a structural drawing of the ultrasonic probe according to the first method. As shown in FIG. 11, in said configuration, layering may be carried out without taking into consideration the shape ratio (w/t) because a single low AI matching layer 5 is layered. Directional characteristics of the ultrasonic transducer 3 deteriorate in the case of the single acoustic matching layer; however, by adopting materials with a high Poisson's ratio as the material (for example, a polyurethane material) for the low AI matching layer 5, deterioration of the directivity may be reduced. Generally, the acoustic impedance value of the upper surface electrode extracting layer 6 is the value between the high AI matching layer 4 and the low AI matching layer 5, so the upper surface electrode extracting layer 6 must be layered on the ultrasonic transducer 3 side of the low AI matching layer 5; however, in this configuration, the ultrasonic transducer 3 to the high AI matching layer 4 is divided and the upper surface electrode extracting layer 6 as well as the low AI matching layer 5 may be layered like sheets on the high AI matching layer 4 side, and by sufficiently ensuring a contact area between the upper surface electrode extracting layer 6 and the high AI matching layer 4, the upper electrode (GND electrode) of the ultrasonic transducer 3 may be extracted with high reliability.

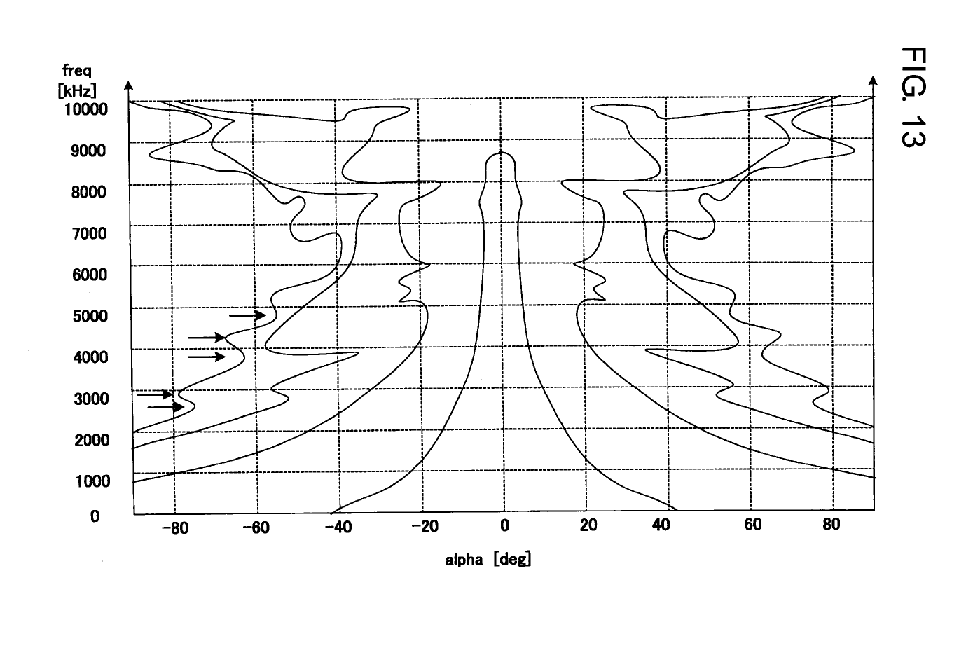

The second method involves dividing the non-rubber low AI matching layer 5 and filling the shaped grooves with rubber materials. FIG. 12 is a structural drawing of the ultrasonic probe according to the second method. In the configuration indicated in FIG. 12, the shape ratio (w/t) of the low AI matching layer 5 cannot be sufficiently reduced; however, the transverse oscillation generated may be reduced with rubber materials filled in the grooves. Moreover, the low AI matching layer 5 is completely or partially divided, so the effects of crosstalk between the elements may be reduced.

### SUMMARY OF THE INVENTION

### Problem to be Solved by the Invention

FIG. 13 is a diagram showing the outcome of a directivity simulation related to conventional technology. In the ultrasonic probe shown in FIG. 11, the low AI matching layer 5 is layered by saddling a plurality of elements, so due to the effect of the crosstalk between elements, as shown in FIG. 13 with an arrow, the directivity of the element finely changes per frequency, and the directivity may become narrow depending on the frequency when rendering images with the ultrasonic diagnostic equipment. Accordingly, the oscillation angle of the ultrasonic beam becomes smaller and causes significant deterioration of the resolution (bearing resolution) in the scanning direction during ultrasonic imaging.

In the ultrasonic probe shown in FIG. 12, when a configuration comprising the upper surface electrode extracting layer 6 of the ultrasonic transducer 3 is adopted in order to divide the low AI matching layer 5, the upper surface electrode extracting layer 6 must be divided in the same manner as the low AI matching layer 5. The cutting spacing of the ultrasonic probe becomes very narrow at approximately 0.2 mm, therefore, the reliability when extracting the upper surface electrode (GND electrode) of each element is declined.

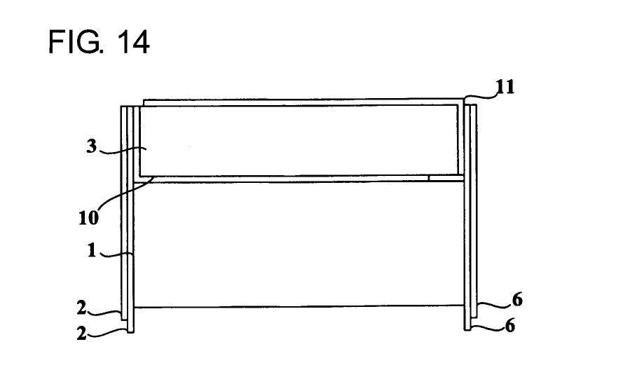

FIG. 14 is a structural drawing of the ultrasonic probe according to conventional examples. As shown in FIG. 14, one method involves extracting from an end of the ultrasonic transducer 3 as another method of extracting the upper surface electrode 11. However, the thickness of the ultrasonic transducer 3 is from 200 μm to 500 μm and is very thin, therefore, sufficiently ensuring the contact surface is difficult. Therefore, there being a problem of low reliability in electrode extraction of the ultrasonic transducer 3.

This embodiment solves the problem mentioned above, with the purpose of providing an ultrasonic probe that prevents deterioration of the bearing resolution in ultrasonic images and further obtains high reliability in electrode extraction of the ultrasonic transducer.

### Means of Solving the Problem

In order to solve the problems mentioned above, the ultrasonic probe of the embodiment comprises an ultrasonic transducer, an electrode extraction layer, and a low acoustic impedance matching layer. The ultrasonic transducer comprises a plurality of elements arranged with predetermined spacing. The electrode extraction layer is electrically connected to the ultrasonic transducer. The sheet-like low acoustic impedance matching layer is provided on the electrode extraction layer, having lower acoustic impedance than the ultrasonic transducer, with the plurality of grooves shaped in parallel in the array direction of elements on the surface of the electrode extraction layer side.

Moreover, the ultrasonic probe of the embodiment comprises an ultrasonic transducer, an electrode extraction layer, and a low acoustic impedance matching layer. The ultrasonic transducer comprises a plurality of electrodes arranged with predetermined spacing. The electrode extraction layer is electrically connected to the ultrasonic transducer. The sheet-like low acoustic impedance matching layer is provided on the electrode extraction layer, wherein, it has smaller acoustic impedance than the ultrasonic transducer with the holes shaped on the surface of the electrode extraction layer side with smaller spacing than the predetermined spacing.

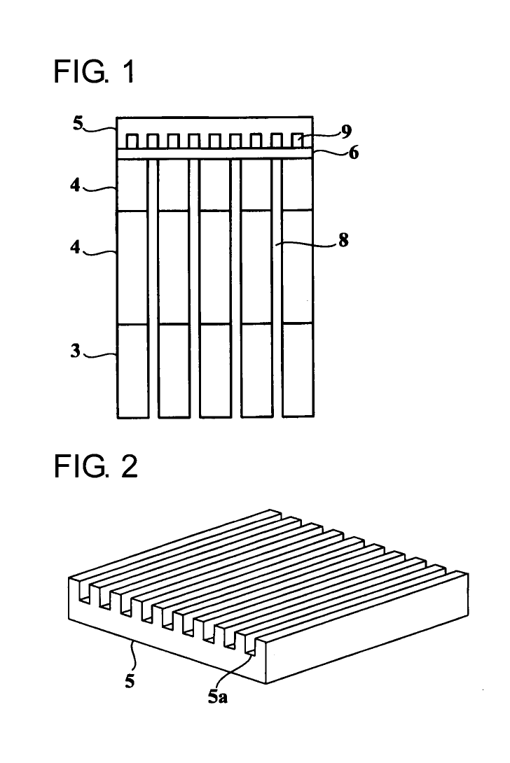

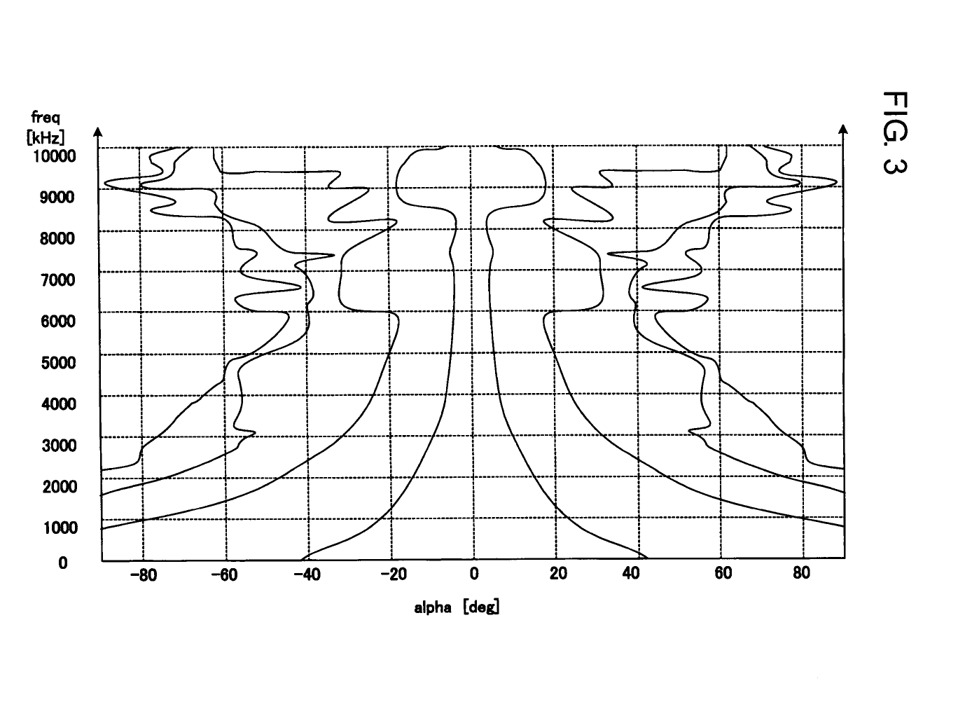

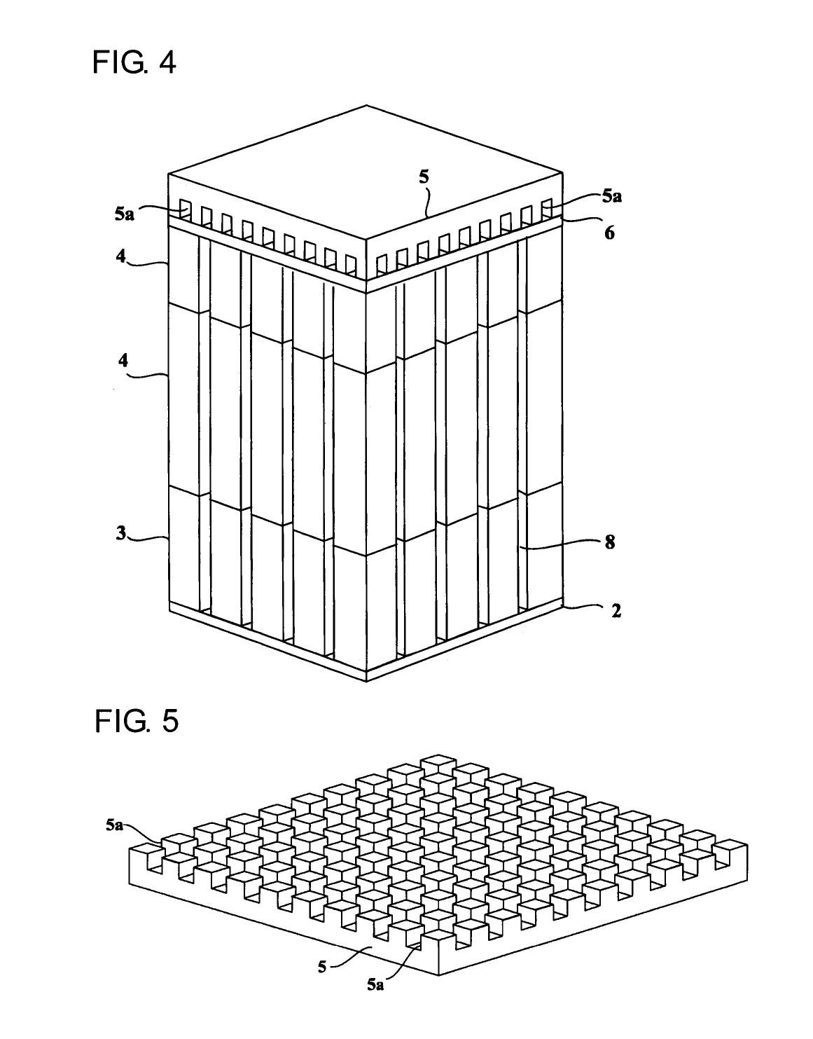

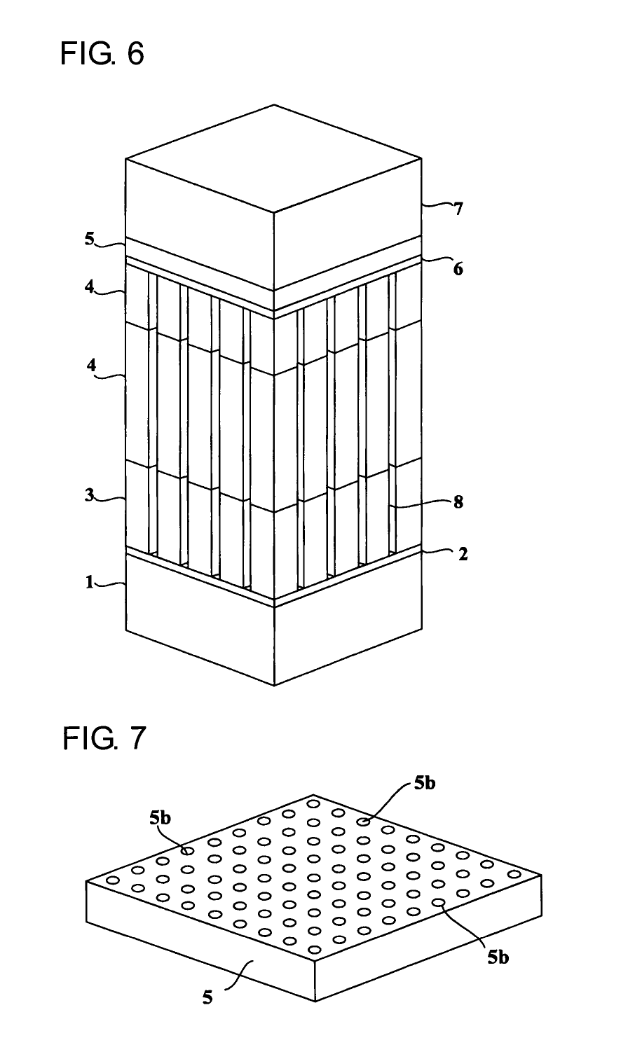

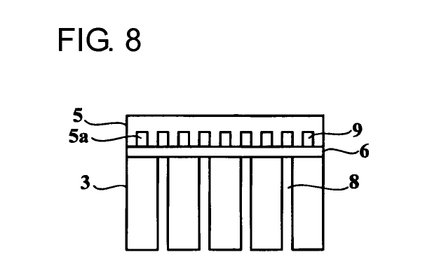

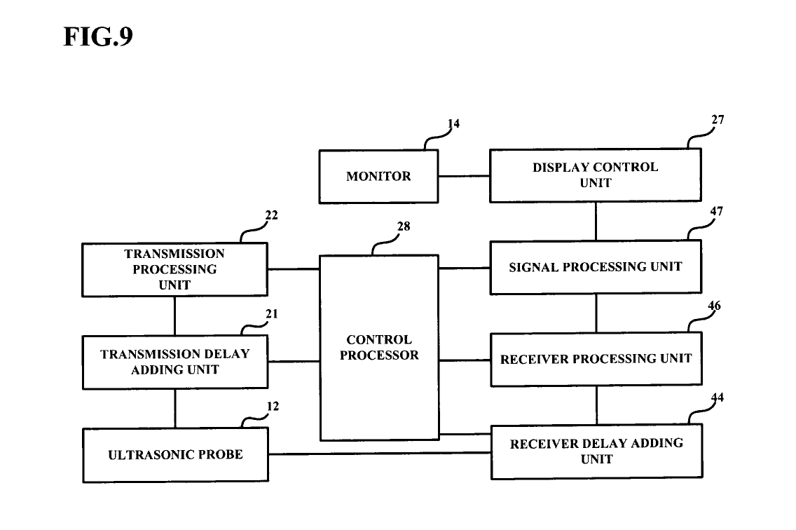

### BRIEF DESCRIPTION OF THE DRAWINGS

[FIG. 1] is an image showing configurations of the ultrasonic transducer, acoustic matching layer, etc. according to Embodiment 1.

[FIG. 2] is a structural diagram of the low AI matching layer.

[FIG. 3] is a diagram showing the simulation results of the directivity of the ultrasonic probe according to Embodiment 1.

[FIG. 4] is a diagram showing configurations of the ultrasonic transducer, acoustic matching layer, etc. according to Embodiment 2.

[FIG. 5] is a structural diagram of the low AI matching layer.

[FIG. 6] is a structural diagram of a typical ultrasonic 2D-array probe.

[FIG. 7] is a structural diagram of the low AI matching layer according to Embodiment 3.

[FIG. 8] is a diagram showing configurations of the ultrasonic transducer, etc. according to Embodiment 4.

[FIG. 9] is a fundamental structural block diagram of the ultrasonic diagnostic equipment.

[FIG. 10] is a fundamental block diagram of the ultrasonic 1D-array probe.

[FIG. 11] is a structural diagram of the ultrasonic probe related to conventional examples.

[FIG. 12] is a structural diagram of the ultrasonic probe related to conventional examples.

[FIG. 13] is a diagram showing the simulation result of the directivity of the ultrasonic probe related to conventional examples.

[FIG. 14] is a structural diagram of the ultrasonic probe related to conventional examples.

### DESCRIPTION OF THE PREFERRED EMBODIMENT

### Embodiment 1

The fundamental configuration of the ultrasonic diagnostic equipment provided with an ultrasonic probe 12 according to Embodiment 1 is described with reference to FIG. 9. FIG. 9 is a fundamental structural block diagram of the ultrasonic diagnostic equipment.

As shown in FIG. 9, the ultrasonic diagnostic equipment is used for diagnosis of diseases of the living body (patient) in the medical field. More specifically, in the ultrasonic diagnostic equipment transmits ultrasonic waves are transmitted inside the subject body by the ultrasonic probe provided with the ultrasonic transducer. Subsequently, the reflected waves of the ultrasonic waves generated from non-conformance of the acoustic impedance inside the subject body are received by the ultrasonic probe, and the internal state of the subject is imaged based on said reflected waves.

The ultrasonic 1D-array probe with a plurality of elements (micro-oscillator) one-dimensionally arranged in array and the ultrasonic 2D-array probe with a plurality of elements two-dimensionally arranged in array are used as ultrasonic diagnostic equipment.

The ultrasonic diagnostic equipment comprises: the ultrasonic probe 12, a transmission delay adding unit 21, a transmission processing unit 22, a control processor (CPU) 28, a receiver delay adding unit 44, a receiver processing unit 46, a signal processing unit 47, a display control unit 27, and a monitor 14.

The ultrasonic probe 12 comprises the ultrasonic transducer, a matching layer, a backing material, etc.

The ultrasonic probe 12 is provided with a plurality of ultrasonic transducers on a known rear material, and the known matching layer is provided on said ultrasonic transducer. That is, these are layered in the order of: the rear material, the ultrasonic transducer, and the matching layer. In the ultrasonic transducer, the surface provided with the matching layer becomes the radiation plane side of the ultrasonic waves, while the opposite surface of said surface (the surface provided with the rear material) becomes the rear surface side. A common (GND) electrode (illustration omitted) is connected to the radiation plane side of the ultrasonic transducer, while a signal electrode (illustration omitted) is connected to the rear surface side.

Acoustic/electric reversible conversion elements, etc., such as a piezoelectric ceramic, etc. may be used as the ultrasonic transducer. For example, ceramic materials such as lead zirconate titanate Pb (Zr, Ti) O₃, lithium niobate (LiNbO₃), barium titanate (BaTiO₃), lead titanate (PbTiO₃), etc. are preferably used.

The ultrasonic transducer generates ultrasonic waves based on driving signals from the transmission processing unit 22. The generated ultrasonic waves are reflected at the surface of discontinuity of the acoustic impedance inside the subject. Each ultrasonic transducer receives said reflected waves, generates signals, and these are taken into the receiver processing unit 46 for each channel.

The acoustic matching layer is provided for better acoustic matching between the acoustic impedance of the ultrasound transducer and the acoustic impedance of the subject. The acoustic matching layer may be comprised of 1 or 2 more layers.

The backing material prevents ultrasonic transmission from the ultrasonic transducer to the rear.

Moreover, among the ultrasonic vibrations oscillated from the ultrasonic transducer and ultrasonic vibrations received, the rear material reduces and absorbs the ultrasonic wave vibration component not necessary for image extraction of the ultrasonic diagnostic equipment. Generally, materials with inorganic particle powders such as tungsten, ferrite, zinc oxide, etc. mixed into synthetic rubber, epoxy resin, or polyurethane rubber, etc. are used as the rear material.

The transmission delay adding unit 21 carries out a delay adding process according to a focal length. The receiver delay adding unit 44 carries out a delay adding process by reverse timing as the delay timing by the transmission delay adding unit 21.

The receiver processing unit 46 comprises: an apodization unit (not illustrated), a frequency modulating/recovering unit (not illustrated), a receiving buffer unit (not illustrated), a receiving mixer (not illustrated), a DBPF (not illustrated), a discrete fourier transformation unit (not illustrated), and a beam memory (not illustrated). The signals are subsequently received at the delayed reception timing and then amplified. The amplified signals are output to the signal processing unit 47.

The signal processing unit 47 comprises an A/D converting circuit, a B-mode processing circuit, a doppler processing circuit, etc.

The A/D converting circuit A/D-converts the signals received by the receiver processing unit 46.

The B-mode processing circuit receives signals from the receiver processing unit 46, and carries out logarithmic amplification, envelope detection processing, etc., to generate data with the signal strength expressed by the brightness of luminance. Said data is transmitted to the display control unit 27 and is displayed on the monitor 14 as a B-mode image in which with the strength of reflected waves expressed by luminance.

The doppler processing circuit analyzes the frequency of speed information based on the signals received from the receiver processing unit 46, extracts the blood flow, tissue, and contrast agent echoing components, and obtains multipoint blood flow information such as average speed, dispersion, power, etc. Particularly, the doppler processing circuit reads multiple-phase recovery data from the receiver processing unit 46, calculates the spectrum obtained in each range, and calculates CW spectrum image data by using these.

The display control unit 27 uses the data received from the signal processing unit 47 to generate ultrasonic images. Furthermore, the display control unit 27 synthesizes the generated images together with character data of various parameters, scales, etc., and outputs these to the monitor 14 as video signals.

The control processor (CPU) 28 includes a function as information processing equipment, and controls the actions of said respective unit. That is, it controls the action of the ultrasonic diagnostic equipment body. The control processor 28 reads an exclusive program for performing a real-time display function of images from the memory and a control program for performing a predetermined scanning sequence, develops these on a memory provided into the control processor, and performs calculation, control, etc. related to the respective processes.

The memory stores a predetermined scanning sequence for collecting a plurality of volume data from different view setting angles, an exclusive program for realizing a real-time display function of images, a control program that carries out image generation and display processing, diagnostic information (patient ID, findings by the doctor, etc.), a diagnostic program, transmitting and receiving conditions, a body mark generating program, and other data groups.

In the above, the fundamental configuration of the ultrasonic diagnostic equipment provided with the ultrasonic probe 12 was described. Next, the main configuration of the ultrasonic probe according to Embodiment 1 is described.

The fundamental configuration of the ultrasonic probe is, as mentioned above, configured from an acoustic lens 7, a high AI matching layer 4, a low AI matching layer 5, an ultrasonic transducer 3, a lower surface electrode extraction layer 2, an upper surface electrode extracting layer 6, and a rear material 1, the subject being contacted to the ultrasonic probe via the acoustic lens 7 (refer to FIG. 10). The ultrasonic transducer 3 is configured such that the plurality of elements are arranged with the predetermined spacing (element pitch) by an array dividing groove 8. The high AI matching layer 4 is also divided by the same spacing as the element pitch by the array dividing groove 8, and in a configuration thereof, the divided matching layers are arranged in the same location as the element (refer to FIG. 11). Each divided matching layer may be referred to as a fragment.

The difference between the ultrasonic probe according to Embodiment 1 and the conventional ultrasonic probe shown in FIG. 11 is the configuration of the low AI matching layer 5.

Next, the configuration of the low AI matching layer 5 is described with reference to FIG. 1. FIG. 1 is a diagram showing configurations of the ultrasonic transducer 3 and the acoustic matching layer, etc. As shown in FIG. 1, the grooves 5a are shaped on the surface of the ultrasonic transducer 3 side of the low AI matching layer 5 (surface adhering to the upper surface electrode extracting layer 6) parallel to the element array direction (element elevation direction) with a spacing of ½ or less of the element pitch. The shaped groove 5a depth is preferably 25% to 75% of the low AI matching layer 5. Moreover, the groove 5a width is preferably ¼ or smaller of the element pitch length. Furthermore, the grooves 5a are preferably filled with the filling agent.

Furthermore, in order to maintain the function of the ultrasonic probe, the low AI matching layer 5 should be shaped with materials having a Poisson's ratio of 0.43 or more, and be shaped from, for example, materials from one among polyurethane, polyethylene, and polyester.

Next, the manufacturing method of the ultrasonic probe is described with reference to FIG. 2. FIG. 2 is the structural diagram of the low AI matching layer. The grooves 5a with ½ or less of the spacing and 25% to 75% of the depth of the thickness of the array dividing groove 8 are shaped on the ultrasonic transducer 3 side of the low AI matching layer 5 before adhesion in a direction parallel to the element array direction (refer to FIG. 2).

By having the groove 5a with ½ or less of the spacing of the array dividing groove 8, the bearing resolution may be further stabilized. Moreover, the groove 5a thickness is made 25% to 75% the thickness of the low AI matching layer 5, thereby allowing the acoustic matching function to be maintained.

Next, said worked surface is adhered to the upper surface electrode extracting layer 6 in the same manner as the conventional method. At this time, the grooves 5a should be parallel to the array dividing groove 8, and do not need to be conformed. Accordingly, if the array dividing grooves 8 and the grooves 5a of the low AI matching layer 5 are uniformly arranged (angular adjustment), adhesion may become relatively easy. Regarding the filling method of the filling agent in the grooves 5a, the filling agent may be filled in advance when shaping the grooves 5a or may be filled with an epoxy adhesive applied during adhesion of the low AI matching layer 5 to the upper surface electrode extracting layer 6. Furthermore, the filling agent and the adhesive may be materials not affecting the acoustic matching function of the low AI matching layer 5. The groove 5a shape may be stabilized by filling the grooves 5a with the filling agent.

FIG. 3 is a diagram showing the outcome of the directivity simulation according to Embodiment 1. As is evident from comparing FIG. 3 and FIG. 13, the element directivity does not finely change with each frequency, and moreover, the directivity is not narrowed due to the frequency when rendering images using the ultrasonic diagnostic equipment. Thereby, the oscillation angle of the ultrasonic beam is not reduced and the resolving power (bearing resolution) in the scanning direction of the ultrasonic image may be prevented from deteriorating.

### Embodiment 2

Next, the ultrasonic probe according to Embodiment 2 is described with reference to FIGS. 4 and 6.

FIG. 4 is the structural diagram of the ultrasonic 2D-array probe according to Embodiment 2, FIG. 5 is the structural diagram of the low AI matching layer, and FIG. 6 is the structural diagram of a general ultrasonic 2D-array probe used for comparison. Furthermore, each part configuring the ultrasonic probe is the same as in Embodiment 1.

As shown in FIGS. 4 and 6, the only difference between the ultrasonic 2D-array probe according to Embodiment 2 and the general ultrasonic 2D-array probe is the configuration of the low AI matching layer 5.

Next, the configuration of the low AI matching layer 5 is described. As shown in FIG. 4, the elements of the ultrasonic 2D-array probe are divided in the element elevation direction and the element azimuth direction in a square-box pattern; therefore, the grooves 5a shaped in the low AI matching layer 5 must also be shaped in a square-box pattern. If the element pitch of the element elevation direction and the element azimuth direction are different, the spacing of the grooves 5a shaped in the low AI matching layer 5 are ½ or less of the spacing of the element pitch of the respective directions (refer to FIG. 5). Here, the element azimuth direction refers to the direction orthogonally intersecting the elevation direction and the layering direction of the acoustic matching layer, respectively.

By means of having the spacing of the grooves 5a of respective directions ½ or less of the spacing of the element pitch deterioration of the bearing resolution in the three-dimensional images may be prevented.

If the angles of the array dividing groove 8 and the grooves 5a of the low AI matching layer 5 are adjusted in the same manner as Embodiment 1, adhesion is relatively easily. In the same manner as Embodiment 1, the shaped grooves 5a are preferably filled with the filling.

### Embodiment 3

Next, the configuration of the ultrasonic probe according to Embodiment 3 is described with reference to FIG. 7. Furthermore, the fundamental configuration of the ultrasonic probe is the same as in Embodiment 1.

FIG. 7 is a structural diagram of the low AI matching layer. As shown in FIG. 7, the holes 5b with a diameter ¼ or smaller the element pitch are arranged at a spacing of ½ or smaller of the element pitch on said upper surface electrode side of the low AI matching layer 5. Thereby, sufficient acoustic pressure may be obtained. In Embodiment 3, holes 5b are provided as an alternative to the grooves 5a of Embodiment 1.

The depth of the shaped holes 5b is preferably 25% to 75% of the matching layer thickness. Moreover, the holes 5b are preferably filled with the filling.

The processing method of the present embodiment is the same as in Embodiment 1 expect for the fact that said grooves 5a were changed to said holes b.

As mentioned above, the effect of crosstalk between elements is reduced according to the present embodiment; therefore, changes in the element directivity for each frequency may be reduced. Thereby, the oscillation angle of the ultrasonic beam may be maintained without depending on the frequency used when rendering images with the ultrasonic diagnostic equipment, and deterioration of the bearing resolution of the ultrasonic images may be prevented. Moreover, due to the configuration of processing and layering the low AI matching layer 5 in advance, the upper surface electrode extracting layer 6 may be layered without dividing and high credibility may be obtained in electrode extraction of the ultrasonic transducer 3.

### Embodiment 4

Next, the configuration of the ultrasonic probe according to Embodiment 4 is described with reference to FIG. 8. Furthermore, in Embodiment 4, configurations differing from Embodiment 1 are mainly described and descriptions thereof are omitted regarding configurations that are the same as in Embodiment 1.

In Embodiment 1, the high AI matching layer 4 is arranged on the ultrasonic transducer 3, the upper surface electrode extracting layer 6 is provided on the high AI matching layer 4, and the low AI matching layer 5 is provided on the upper surface electrode extracting layer 6.

In contrast, configurations of the ultrasonic transducer 3, etc. of Embodiment 4 are described with reference to FIG. 8. FIG. 8 is a diagram showing the configurations of the ultrasonic transducer 3, etc. As shown in FIG. 8, the upper surface electrode extracting layer 6 is provided on the ultrasonic transducer 3 and the low AI matching layer 5 is provided on the upper surface electrode extracting layer 6.

Furthermore, in Embodiment 1, the low AI matching layer 5 had lower impedance than the high AI matching layer 4; however, in Embodiment 4, the low AI matching layer 5 has lower acoustic impedance than the ultrasonic transducer 3.

The high AI matching layer 4 may be omitted in Embodiment 4 because when the ultrasonic transducer 3 is made with materials having a small acoustic impedance difference for the subject, interpositioning two types of the high AI matching layer 4 and the low AI matching layer 5 between the ultrasonic transducer 3 and the subject is not necessary, and it is only necessary to interposition the low AI matching layer 5 is sufficient.

Furthermore, in Embodiment 4, in the same manner as Embodiment 1, the array dividing groove 8 is provided in the ultrasonic transducer 3 and the grooves 5a are provided in the low AI matching layer 5. Furthermore, the grooves 5a are preferably filled with the filling 9.

Moreover, in Embodiment 4, the holes 5b may be provided instead of the grooves 5a in the same manner as Embodiment 3.

Several embodiments of the present invention were explained; however, said embodiments were presented as examples and are not intended to limit the range of the invention. Said new embodiments may be carried out in other various forms, and various abbreviations, revisions, and changes may be carried out in a range not deviating from the gist of the invention. These embodiments and deformations thereof are included in the range and gist of the invention and additionally included in the invention described in the patent claims and the equivalent thereof.

### EXPLANATION OF SYMBOLS

1 Rear material

2 Lower surface electrode extraction layer

3 Ultrasonic transducer

4 High AI matching layer

5 Low AI matching layer

5a Grooves

5b Holes

6 Upper surface electrode extracting layer

7 Acoustic lens

8 Array dividing groove

9 Filling

10 Lower surface electrode

11 Upper surface electrode

1. An ultrasonic probe, comprising:
an ultrasonic transducer comprising a plurality of elements arranged with predetermined spacing,
an electrode extraction layer electrically connected to said ultrasonic transducer, and
a sheet-like low acoustic impedance matching layer provided on said electrode extraction layer, having lower acoustic impedance than said ultrasonic transducer, wherein; a plurality of grooves are shaped in parallel in the array direction of said elements on the surface of said electrode extraction layer side.

2. The ultrasonic probe, comprising:
the ultrasonic transducer comprising a plurality of elements arranged with predetermined spacing,
the electrode extraction layer electrically connected to said ultrasonic transducer, and
the sheet-like low acoustic impedance matching layer provided on said electrode extraction layer, having lower acoustic impedance than said ultrasonic transducer, wherein; holes with smaller spacing than said predetermined spacing are shaped on the surface of said electrode extraction layer side.

3. The ultrasonic probe according to claim 1, further comprising:
a high acoustic impedance matching layer comprising a fragment arranged on said ultrasonic transducer with the same spacing as said predetermined spacing, and an acoustic impedance lower than said ultrasonic transducer and higher than said low acoustic impedance matching layer, wherein:
said electrode extraction layer is provided on said high acoustic impedance matching layer.

4. The ultrasonic probe according to claim 1, wherein:
said plurality of grooves are arranged at approximately ½ or less of the spacing of said predetermined spacing.

5. The ultrasonic probe according to claim 3, wherein:
said ultrasonic transducer and said high acoustic impedance matching layer are arranged in a two-dimensional direction, and
said plurality of grooves are arranged in parallel with respect to said two-dimensional direction.

6. The ultrasonic probe according to claim 2, wherein:
said hole diameter corresponds to approximately ¼ or less of the length of said predetermined spacing.

7. The ultrasonic probe according to claim 1, wherein:
the thickness of said low acoustic impedance matching layer is approximately ¼ or less of the ultrasonic wavelength, and
said groove depth is 25% to 75% of the thickness of said low acoustic impedance matching layer.

8. The ultrasonic probe according to claim 2, wherein:
the thickness of said low acoustic impedance matching layer is approximately ¼ of the ultrasonic wavelength and
said hole depth is 25% to 75% of the thickness of said low acoustic impedance matching layer.

9. The ultrasonic probe according to claim 1, wherein:
said grooves are filled with a filling agent.

10. The ultrasonic probe according to claim 2, wherein:
said holes are filled with a filling agent.

11. The ultrasonic probe according to claim 9, wherein:
said filling agent is an epoxy adhesive for adhering said low acoustic impedance matching layer and the electrode extraction layer.

12. The ultrasonic probe according to claim 1, wherein:
said low acoustic impedance matching layer is shaped from materials having a Poisson's ratio of 0.43 or greater.

13. The ultrasonic probe according to claim 1, wherein:
said low acoustic impedance matching layer is shaped from one material among polyurethane, polyethylene, and polyester.
